(19) United States
(12) Patent Application Publication
(10) Pub. No.: US 2013/0292283 A1
(43) Pub. Date: Nov. 7, 2013

(54) DEFIBRILLATOR CABINET WITH VIBRATION ISOLATION

(71) Applicant: PHYSIO-CONTROL, INC., Redmond, WA (US)

(72) Inventors: John Robert Knapinski, Kirkland, WA (US); Gregory T. Kavounas, Bellevue, WA (US); Oscar Hernandez Rojas, Bothell, WA (US)

(73) Assignee: Physio-Control, Inc., Redmond, WA (US)

(21) Appl. No.: 13/887,176

(22) Filed: May 3, 2013

Related U.S. Application Data
(60) Provisional application No. 61/642,392, filed on May 3, 2012.

(51) Int. Cl. A61B 19/02

(52) U.S. Cl. CPC A61B 19/02
USPC 206/363

(57) ABSTRACT

An apparatus for storing a defibrillator, such as an AED, on a host structure includes a container shell mountable to the host structure. The container shell may be used to store the defibrillator within it. Also included in the container shell is a vibration-dampening material disposed between the host structure and the housing of the defibrillator. The vibration-dampening material is configured to reduce an amount of vibration of the host structure imparted to the defibrillator. This is especially useful for storing AEDs on means of transportation, i.e. where the traveling host structure is a bus, an airplane, a ship, or an elevator, and where the vibration sources from its propulsion system.

### FIELD

This invention generally relates to external defibrillators, and particularly to defibrillator storage devices.

### BACKGROUND

In humans, the heart beats to sustain life. In normal operation, it pumps blood through the various parts of the body. More particularly, the various chamber of the heart contract and expand in a periodic and coordinated fashion, which causes the blood to be pumped regularly. More specifically, the right atrium sends deoxygenated blood into the right ventricle. The right ventricle pumps the blood to the lungs, where it becomes oxygenated, and from where it returns to the left atrium. The left atrium pumps the oxygenated blood to the left ventricle. The left ventricle, then, expels the blood, forcing it to circulate to the various parts of the body.

The heart chambers pump because of the heart's electrical control system. More particularly, the sinoatrial (SA) node generates an electrical impulse, which generates further electrical signals. These further signals cause the above-described contractions of the various chambers in the heart, in the correct sequence. The electrical pattern created by the sinoatrial (SA) node is called a sinus rhythm.

Sometimes, however, the electrical control system of the heart malfunctions, which can cause the heart to beat irregularly, or not at all. The cardiac rhythm is then generally called an arrhythmia. Arrhythmias may be caused by electrical activity from locations in the heart other than the SA node. Some types of arrhythmia may result in inadequate blood flow, thus reducing the amount of blood pumped to the various parts of the body. Some arrhythmias may even result in a Sudden Cardiac Arrest (SCA). In a SCA, the heart fails to pump blood effectively, and, if not treated, death can occur. In fact, it is estimated that SCA results in more than 250,000 deaths per year in the United States alone. Further, a SCA may result from a condition other than an arrhythmia

One type of arrhythmia associated with SCA is known as Ventricular Fibrillation (VF). VF is a type of malfunction where the ventricles make rapid, uncoordinated movements, instead of the normal contractions. When that happens, the heart does not pump enough blood to deliver enough oxygen to the vital organs. The person's condition will deteriorate rapidly and, if not reversed in time, they will die soon, e.g. within ten minutes.

Ventricular Fibrillation can often be reversed using a life-saving device called a defibrillator. A defibrillator, if applied properly, can administer an electrical shock to the heart. The shock may terminate the VF, thus giving the heart the opportunity to resume pumping blood. If VF is not terminated, the shock may be repeated, often at escalating energies.

A challenge with defibrillation is that the electrical shock must be administered very soon after the onset of VF. There is not much time: the survival rate of persons suffering from VF decreases by about 10% for each minute the administration of a defibrillation shock is delayed. After about 10 minutes the rate of survival for SCA victims averages less than 2%.

The challenge of defibrillating early after the onset of VF is being met in a number of ways. First, for some people who are considered to be at a higher risk of VF or other heart arrhythmias, an Implantable Cardioverter Defibrillator (ICD) can be implanted surgically. An ICD can monitor the person's heart, and administer an electrical shock as needed. As such, an ICD reduces the need to have the higher-risk person be monitored constantly by medical personnel.

Regardless, VF can occur unpredictably, even to a person who is not considered at risk. As such, VF can be experienced by many people who lack the benefit of ICD therapy. When VF occurs to a person who does not have an ICD, they collapse, because blood flow has stopped. They should receive therapy quickly.

For a VF victim without an ICD, a different type of defibrillator can be used, which is called an external defibrillator. External defibrillators have been made portable, so they can be brought to a potential VF victim quickly enough to revive them.

During VF, the person's condition deteriorates, because the blood is not flowing to the brain, heart, lungs, and other organs. Blood flow must be restored, if resuscitation attempts are to be successful.

Cardiopulmonary Resuscitation (CPR) is one method of forcing blood flow in a person experiencing cardiac arrest. In addition, CPR is the primary recommended treatment for some patients with some kinds of non-VF cardiac arrest, such as asystole and pulseless electrical activity (PEA). CPR is a combination of techniques that include chest compressions to force blood circulation, and rescue breathing to force respiration.

Properly administered CPR provides oxygenated blood to critical organs of a person in cardiac arrest, thereby minimizing the deterioration that would otherwise occur. As such, CPR can be beneficial for persons experiencing VF, because it slows the deterioration that would otherwise occur while a defibrillator is being retrieved. Indeed, for patients with an extended down-time, survival rates are higher if CPR is administered prior to defibrillation.

Advanced medical devices can actually coach a rescuer who performs CPR. For example, a medical device can issue instructions, and even prompts, for the rescuer to perform CPR more effectively.

It is important that defibrillators work when they are needed. This is especially true when a defibrillator is in a remote location, such as away from a hospital, and there may be no backup defibrillators available. Defibrillators may fail for a variety of reasons. For example, defibrillator batteries may not store enough charge to generate a sufficient shocking force to restart or pace the heart. Other defibrillators may include pads or other parts subject to aging that have deteriorated so much they are not effective.

Often defibrillators are stored in enclosures or container shells such as cabinets. Most cabinets for storing defibrillators are stationary and fixed to a supporting structure, such as a wall of a building. In some instances, however, a cabinet may be subject to occasional or even constant vibration. For example, the cabinet may be mounted to a wall that is subject to vibration, such as a ship, airplane, or elevator. Because cabinets are generally rigid, the wall vibration is also transmitted to the cabinet.

Motion or vibration can cause electrical devices to wear prematurely, defibrillators included. Sudden, repetitive, and/or prolonged motion may cause components within the defibrillator to shift, move, or become loose, and thus lose function. For example solder joints holding electrical components to a circuit board may crack or break because of the stress of motion, causing the components to perform at a level less than optimum, or even not perform at all. The worst time to learn that a defibrillator is non-functional is when it is needed in an emergency.

Embodiments of the invention address these and other limitations of the prior art.

### BRIEF SUMMARY

The present description gives instances of devices, illustrated by embodiments thereof, the use of which may help overcome problems and limitations of the prior art.

In one embodiment, an apparatus for storing a defibrillator on a host structure includes a container shell mountable to the host structure. The container shell may be used to store the defibrillator within it. Also included in the container shell is a vibration-dampening material disposed between the host structure and the housing of the defibrillator. The vibration-dampening material is configured to reduce an amount of vibration of the host structure imparted to the defibrillator.

An advantage over the prior art is that minimizing vibration, of both the sudden and the constant or continuous type, may prolong the life of defibrillators stored within the cabinet.

The apparatus of the invention can be used to store a defibrillator on any host structure, such as a building, as buildings are subject to vibrations. Particular usefulness may be found when storing AEDs on means of transportation, i.e. where the traveling host structure is a bus, an airplane, a ship, or an elevator, and where the vibration sources from the propulsion system of the host structure.

These and other features and advantages of this description will become more readily apparent from the following Detailed Description, which proceeds with reference to the drawings, in which:

### BRIEF DESCRIPTION OF THE DRAWINGS

FIG. 1 is a diagram of a scene where an external defibrillator is used to save the life of a person according to embodiments.

FIG. 2 is a table listing the two main types of external defibrillator, such as the one shown in FIG. 1.

FIG. 3 is a diagram showing components of an external defibrillator, such as the one shown in FIG. 1, which includes components that may be subject to vibration.

FIG. 4 is a front view of a conventional cabinet that stores an external defibrillator.

FIG. 5 is a side-sectional view of a container shell that stores an external defibrillator on vibration-dampening material and is mounted on a host structure according to embodiments of the invention.

FIG. 6 is a front-sectional view of the defibrillator container shell of FIG. 2 according to embodiments.

FIG. 7 is another front-sectional view of the defibrillator container shell of FIG. 2 including a vibration dampening material that receives an external defibrillator substantially matingly according to other embodiments.

FIG. 8 is a side-sectional view of the defibrillator container shell of FIG. 2 mounted on a host structure and including a hook with vibration dampening material according to embodiments of the invention.

FIG. 9 is a side sectional view of a container shell that stores an external defibrillator including a vibration dampening material disposed between the container shell and a host structure according to embodiments of the invention.

FIG. 10 is a side sectional view of the defibrillator container shell of FIG. 6 in which the container shell is affixed to the host structure with a fastener and grommet according to embodiments of the invention.

### DETAILED DESCRIPTION

As has been mentioned, the present description is about a defibrillator cabinet that includes vibration isolation. Embodiments are now described in more detail.

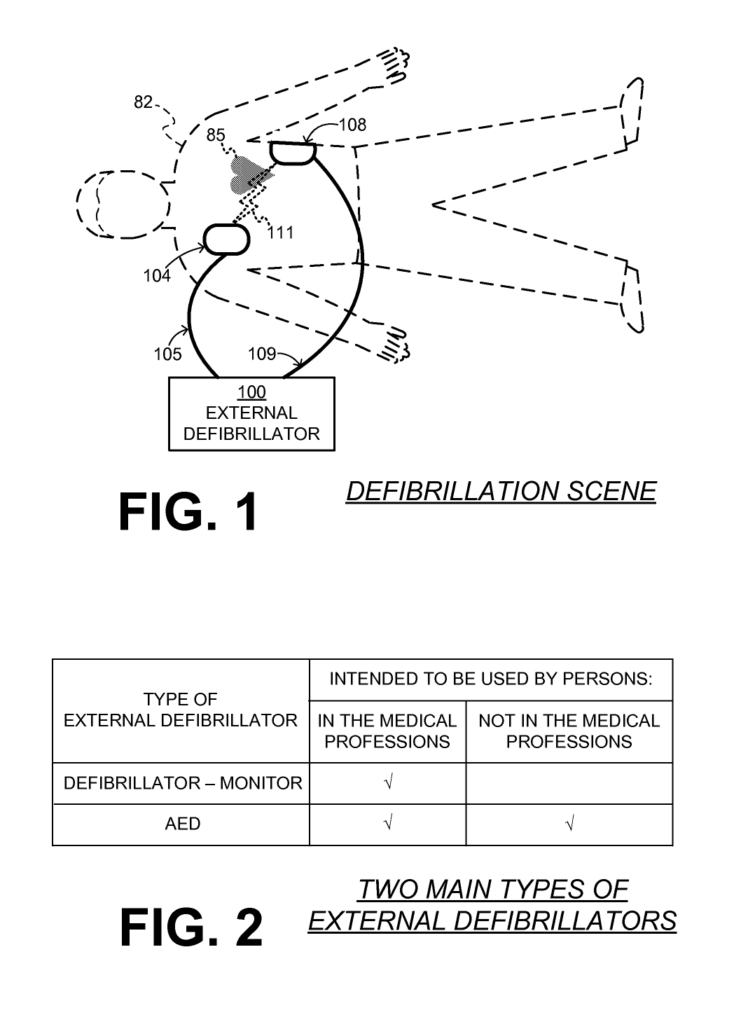

FIG. 1 is a diagram of a defibrillation scene. A person 82 is lying on their back. Person 82 could be a patient in a hospital, or someone found unconscious, and then turned to be on their back. Person 82 is experiencing a condition in their heart 85, which could be Ventricular Fibrillation (VF).

A portable external defibrillator 100 has been brought close to person 82. At least two defibrillation electrodes 104, 108 are usually provided with external defibrillator 100, and are sometimes called electrodes 104, 108. Electrodes 104, 108 are coupled with external defibrillator 100 via respective electrode leads 105, 109. A rescuer (not shown) has attached electrodes 104, 108 to the skin of person 82. Defibrillator 100 is administering, via electrodes 104, 108, a brief, strong electric pulse 111 through the body of person 82. Pulse 111, also known as a defibrillation shock, goes also through heart 85, in an attempt to restart it, for saving the life of person 82.

Defibrillator 100 can be one of different types, each with different sets of features and capabilities. The set of capabilities of defibrillator 100 is determined by planning who would use it, and what training they would be likely to have. Examples are now described.

FIG. 2 is a table listing two main types of external defibrillators, and who they are primarily intended to be used by. A first type of defibrillator 100 is generally called a defibrillator-monitor, because it is typically formed as a single unit in combination with a patient monitor. A defibrillator-monitor is sometimes called monitor-defibrillator. A defibrillator-monitor is intended to be used by persons in the medical professions, such as doctors, nurses, paramedics, emergency medical technicians, etc. Such a defibrillator-monitor is intended to be used in a pre-hospital or hospital scenario.

As a defibrillator, the device can be one of different varieties, or even versatile enough to be able to switch among different modes that individually correspond to the varieties. One variety is that of an automated defibrillator, which can determine whether a shock is needed and, if so, charge to a predetermined energy level and instruct the user to administer the shock. Another variety is that of a manual defibrillator, where the user determines the need and controls administering the shock.

As a patient monitor, the device has features additional to what is minimally needed for mere operation as a defibrillator. These features can be for monitoring physiological indicators of a person in an emergency scenario. These physiological indicators are typically monitored as signals. For example, these signals can include a person's full ECG (electrocardiogram) signals, or impedance between two electrodes. Additionally, these signals can be about the person's temperature, non-invasive blood pressure (NIBP), arterial oxygen saturation/pulse oximetry (SpO2), the concentration or partial pressure of carbon dioxide in the respiratory gases, which is also known as capnography, and so on. These signals can be further stored and/or transmitted as patient data.

A second type of external defibrillator 100 is generally called an AED, which stands for “Automated External Defibrillator”. An AED typically makes the shock/no shock determination by itself, automatically. Indeed, it can sense enough physiological conditions of the person 82 via only the shown defibrillation electrodes 104, 108 of FIG. 1. In its present embodiments, an AED can either administer the shock automatically, or instruct the user to do so, e.g. by pushing a button. Being of a much simpler construction, an AED typically costs much less than a defibrillator-monitor. As such, it makes sense for a hospital, for example, to deploy AEDs at its various floors, in case the more expensive defibrillator-monitor is more critically being deployed at an Intensive Care Unit, and so on.

AEDs, however, can also be used by people who are not in the medical profession. More particularly, an AED can be used by many professional first responders, such as policemen, firemen, etc. Even a person with only first-aid training can use one. And AEDs increasingly can supply instructions to whoever is using them.

AEDs are thus particularly useful, because it is so critical to respond quickly, when a person suffers from VF. Indeed, the people who will first reach the VF sufferer may not be in the medical professions.

Increasing awareness has resulted in AEDs being deployed in public or semi-public spaces, so that even a member of the public can use one, if they have obtained first aid and CPR/AED training on their own initiative. This way, defibrillation can be administered soon enough after the onset of VF, to hopefully be effective in rescuing the person.

There are additional types of external defibrillators, which are not listed in FIG. 2. For example, a hybrid defibrillator can have aspects of an AED, and also of a defibrillator-monitor. A usual such aspect is additional ECG monitoring capability.

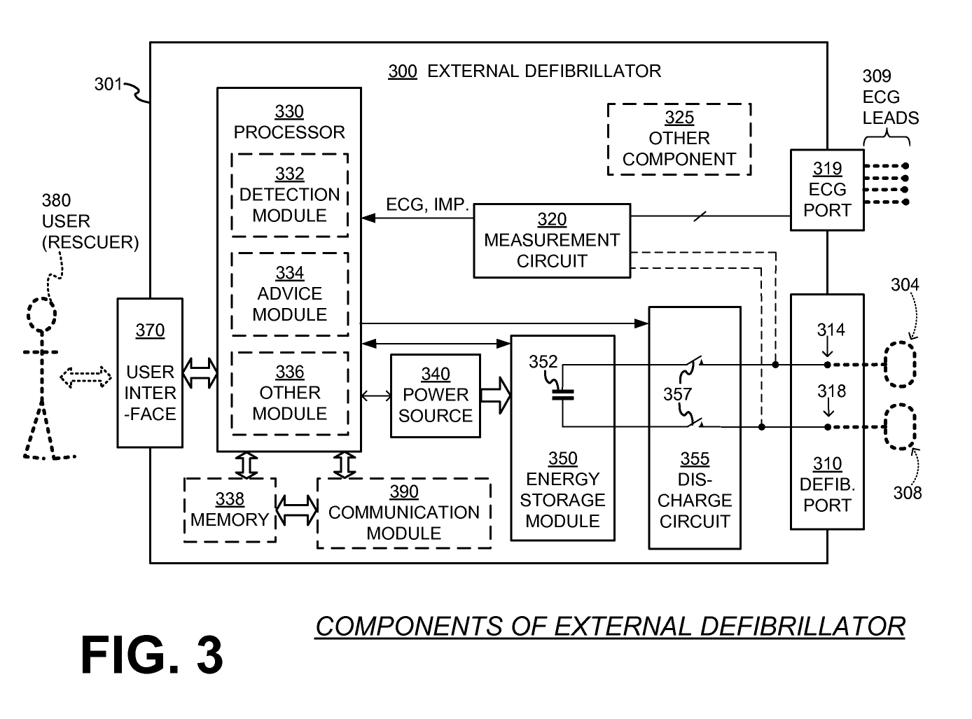

FIG. 3 is a diagram showing components of an external defibrillator 300 made according to embodiments. These components can be, for example, in external defibrillator 100 of FIG. 1. Plus, these components of FIG. 3 can be provided in a housing 301, which is also known as casing 301.

External defibrillator 300 is intended for use by a user 380, who would be the rescuer. Defibrillator 300 typically includes a defibrillation port 310, such as a socket in housing 301. Defibrillation port 310 includes nodes 314, 318. Defibrillation electrodes 304, 308, which can be similar to electrodes 104, 108, can be plugged in defibrillation port 310, so as to make electrical contact with nodes 314, 318, respectively. It is also possible that electrodes can be connected continuously to defibrillation port 310, etc. Either way, defibrillation port 310 can be used for guiding via electrodes to person 82 an electrical charge that has been stored in defibrillator 300, as will be seen later in this document.

If defibrillator 300 is actually a defibrillator-monitor, as was described with reference to FIG. 2, then it will typically also have an ECG port 319 in housing 301, for plugging in ECG leads 309. ECG leads 309 can help sense an ECG signal, e.g. a 12-lead signal, or from a different number of leads. Moreover, a defibrillator-monitor could have additional ports (not shown), and an other component 325 for the above described additional features, such as patient signals.

Defibrillator 300 also includes a measurement circuit 320. Measurement circuit 320 receives physiological signals from ECG port 319, and also from other ports, if provided. These physiological signals are sensed, and information about them is rendered by circuit 320 as data, or other signals, etc.

If defibrillator 300 is actually an AED, it may lack ECG port 319. Measurement circuit 320 can obtain physiological signals through nodes 314, 318 instead, when defibrillation electrodes 304, 308 are attached to person 82. In these cases, a person's ECG signal can be sensed as a voltage difference between electrodes 304, 308. Plus, impedance between electrodes 304, 308 can be sensed for detecting, among other things, whether these electrodes 304, 308 have been inadvertently disconnected from the person.

Defibrillator 300 also includes a processor 330. Processor 330 may be implemented in any number of ways. Such ways include, by way of example and not of limitation, digital and/or analog processors such as microprocessors and digital-signal processors (DSPs); controllers such as microcontrollers; software running in a machine; programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 330 can be considered to have a number of modules. One such module can be a detection module 332, which senses outputs of measurement circuit 320. Detection module 332 can include a VF detector. Thus, the person's sensed ECG can be used to determine whether the person is experiencing VF.

Another such module in processor 330 can be an advice module 334, which arrives at advice based on outputs of detection module 332. Advice module 334 can include a Shock Advisory Algorithm, implement decision rules, and so on. The advice can be to shock, to not shock, to administer other forms of therapy, and so on. If the advice is to shock, some external defibrillator embodiments merely report that to the user, and prompt them to do it. Other embodiments further execute the advice, by administering the shock. If the advice is to administer CPR, defibrillator 300 may further issue prompts for it, and so on.

Processor 330 can include additional modules, such as module 336, for other functions. In addition, if other component 325 is indeed provided, it may be operated in part by processor 330, etc. In some embodiments, the other component 325 or any of the other components within the defibrillator 300 may have a diminished capacity, or even stop working entirely, if the defibrillator 300 is subject to a large vibration or prolonged exposure to vibration.

Defibrillator 300 optionally further includes a memory 338, which can work together with processor 330. Memory 338 may be implemented in any number of ways. Such ways include, by way of example and not of limitation, nonvolatile memories (NVM), read-only memories (ROM), random access memories (RAM), any combination of these, and so on. Memory 338, if provided, can include programs for processor 330, and so on. The programs can be operational for the inherent needs of processor 330, and can also include protocols and ways that decisions can be made by advice module 334. In addition, memory 338 can store prompts for user 380, etc. Moreover, memory 338 can store patient data.

Defibrillator 300 may also include a power source 340. To enable portability of defibrillator 300, power source 340 typically includes a battery. Such a battery is typically implemented as a battery pack, which can be rechargeable or not. Sometimes, a combination is used, of rechargeable and non-rechargeable battery packs. Other embodiments of power source 340 can include AC power override, for where AC power will be available, and so on. In some embodiments, power source 340 is controlled by processor 330.

Defibrillator 300 additionally includes an energy storage module 350. Module 350 is where some electrical energy is stored, when preparing it for sudden discharge to administer a shock. Module 350 can be charged from power source 340 to the right amount of energy, as controlled by processor 330. In typical implementations, module 350 includes one or more capacitors 352, and so on.

Defibrillator 300 moreover includes a discharge circuit 355. Circuit 355 can be controlled to permit the energy stored in module 350 to be discharged to nodes 314, 318, and thus also to defibrillation electrodes 304, 308. Circuit 355 can include one or more switches 357. Those can be made in a number of ways, such as by an H-bridge, and so on.

Defibrillator 300 further includes a user interface 370 for user 380. User interface 370 can be made in any number of ways. For example, interface 370 may include a screen, to display what is detected and measured, provide visual feedback to the rescuer for their resuscitation attempts, and so on. Interface 370 may also include a speaker, to issue voice prompts, etc. Interface 370 may additionally include various controls, such as pushbuttons, keyboards, and so on. In addition, discharge circuit 355 can be controlled by processor 330, or directly by user 380 via user interface 370, and so on.

Defibrillator 300 can optionally include other components. For example, a communication module 390 may be provided for communicating with other machines. Such communication can be performed wirelessly, or via wire, or by infrared communication, and so on. This way, data can be communicated, such as patient data, incident information, therapy attempted, CPR performance, and so on.

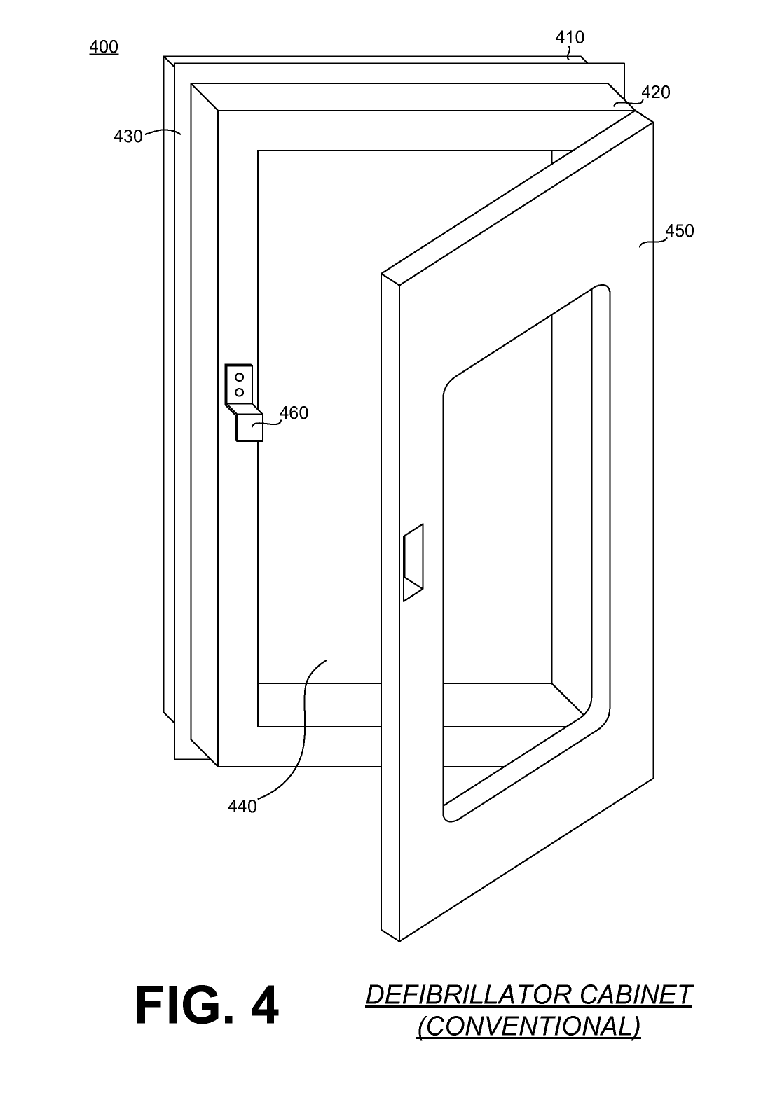

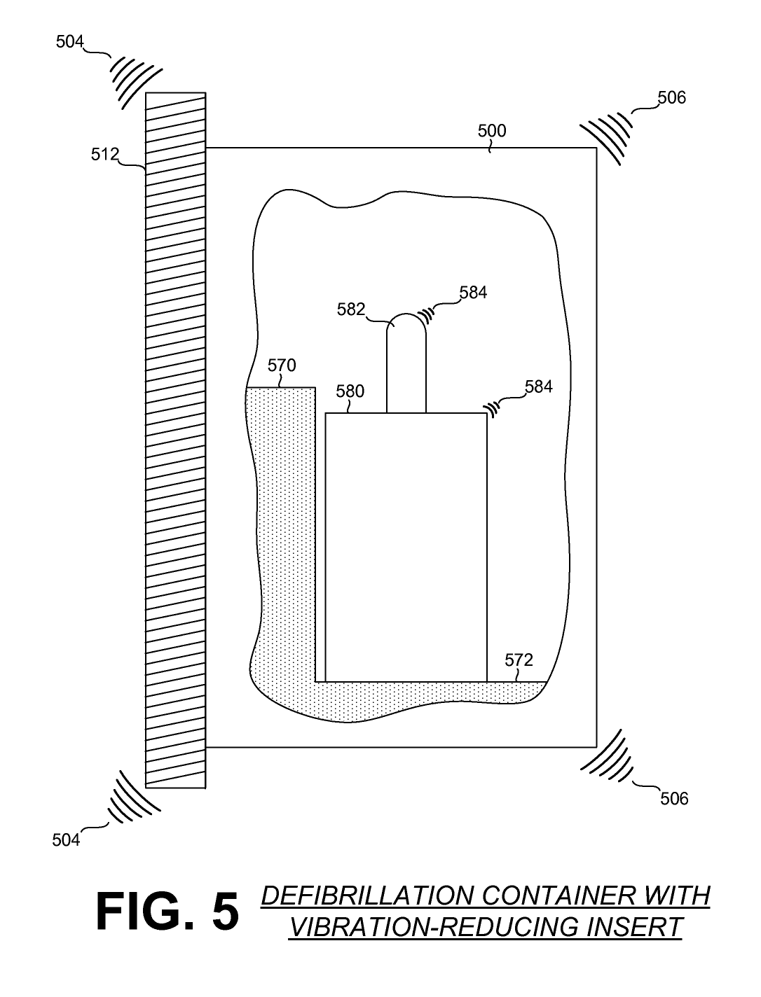

FIG. 4 is a front view of a conventional cabinet 400 that stores an external defibrillator. The cabinet 400 generally includes two portions, a portion 410 that is mounted within a wall opening (not shown), and a portion 420 that extends beyond a wall surface when the cabinet is mounted in the wall opening. A flange 430 extends beyond the cavity and is used to secure the cabinet 400 to the wall, just outside the cavity. The flange 430 may include holes, not illustrated, through which screws or other fasteners may be used to secure the cabinet 400 in place. In other embodiments, the cabinet 400 may be flush mounted to a wall that may not have an opening, and the portion 410 is not present or configured differently. An inner cavity 440 of the cabinet 400 is used to store a defibrillator (not illustrated). The defibrillator may sit on a floor of the inner cavity 440 or be hung on a hook, for example. A door 450 encloses the cavity 400 and may be secured by a latch or other closing mechanism 460. As described above, the cabinet may be mounted on a wall of a building, or on a wall of something that moves, such as an elevator, ship, airplane, or bus.

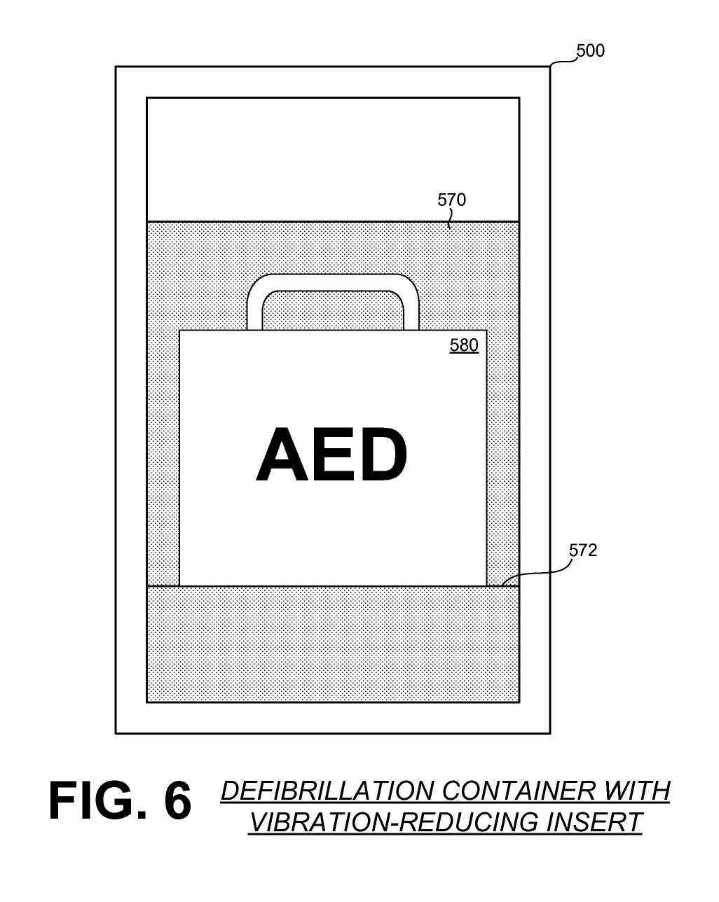

FIG. 5 is a side-sectional view and FIG. 6 is a front view of a container shell or cabinet 500 made according to embodiments of the invention. Container shell or cabinet 500 stores an external defibrillator 580 on vibration-dampening material 570, and is mounted on a host structure 512. More specifically, embodiments of the invention are directed to an apparatus for storing, on a host structure 512, such as a wall that is subject to vibration, an external defibrillator 580 that has a housing, such as housing 301 in FIG. 3. The apparatus includes a container shell 500 mountable to the host structure 512, and is structured to store the defibrillator 580 within. The apparatus also includes a vibration-dampening material 570 disposed between the host structure 512 and the housing of the defibrillator 580. The vibration-dampening material 570 is configured to reduce an amount of vibration of the host structure 512 imparted to the defibrillator 580.

In FIG. 5 vibration of the host structure 512 is illustrated as 504. Depending on the environment, the vibration 504 may be caused by elevator motion, or vehicle motion such as a bus, ship, or airplane. In other environments the host structure 512 may be in an area where earthquakes are present, and the vibration 504 may be caused by an earthquake or weather events such as thunder. In yet other environments the host structure 512 may be proximate a road or railroad and may vibrate when heavy vehicles or trains pass nearby. Because the container shell 500 is typically attached or affixed to the host structure 512, the vibration 504 of the host structure is imparted to the container shell, which is illustrated as 506, and therefore the vibration 506 will be similar in magnitude to the vibration 504.

In the embodiment illustrated in FIG. 5, the vibration dampening material 570 is located or disposed within the container 500, and provides a resting surface 572. The resting surface 572 is in physical contact with the housing of the defibrillator 580 for supporting the defibrillator. The resting surface 572 is more clearly illustrated in FIG. 6.

Additionally, the vibration dampening material 570 may include access passages to mounting holes through which the container shell 500 may be affixed to the host structure 512. In some embodiments the vibration dampening material 570 partially fills the container shell 500 while in others it substantially fills the container shell.

The vibration dampening material 570 dampens, absorbs completely, or at least reduces the amount of container vibration 506 imparted to the defibrillator 580. As a result, the defibrillator 580 will experience less vibration, illustrated as 584 than the vibration 506 of the container shell 500. Accordingly, components of the defibrillator 580 are protected, to at least some degree, from the vibration 506 of the container shell 500. This reduced vibration protects the components of the defibrillator 580, and may reduce the potential that the defibrillator 580 malfunctions due to strong, repetitive, and/or prolonged vibration of the host structure that cabinet 500 is attached to.

Referring back to the embodiment of FIG. 5, the vibration dampening material 570 and the container shell 500 are arranged such that the vibration 504 travels from the host structure 512 first to the container shell, and then from the container shell to the vibration dampening material. More specifically, in such an embodiment, the vibration-dampening material is positioned between the housing of the defibrillator 580 and the container shell 500, although other arrangements are possible, as described in more detail below.

The vibration material 570 may be made of foam or other suitable material. The particular material used for the vibration dampening material may be selected based on factors such as stiffness, durability, vibration absorption, softness, density, availability, and cost. Moreover, where the host is a means of transportation, the choice of the material may be made in view of the environment where the host travels.

In some embodiments, the vibration material 570 may be removable from the container shell 500 so that it may be replaced. The feature of removability may be particularly well suited for retro-fitting existing container shells, if everything fits, etc. The feature of removability may also be useful if the material 570 loses some of its attributes over time due to environmental factors, for example if the host travels by sea.

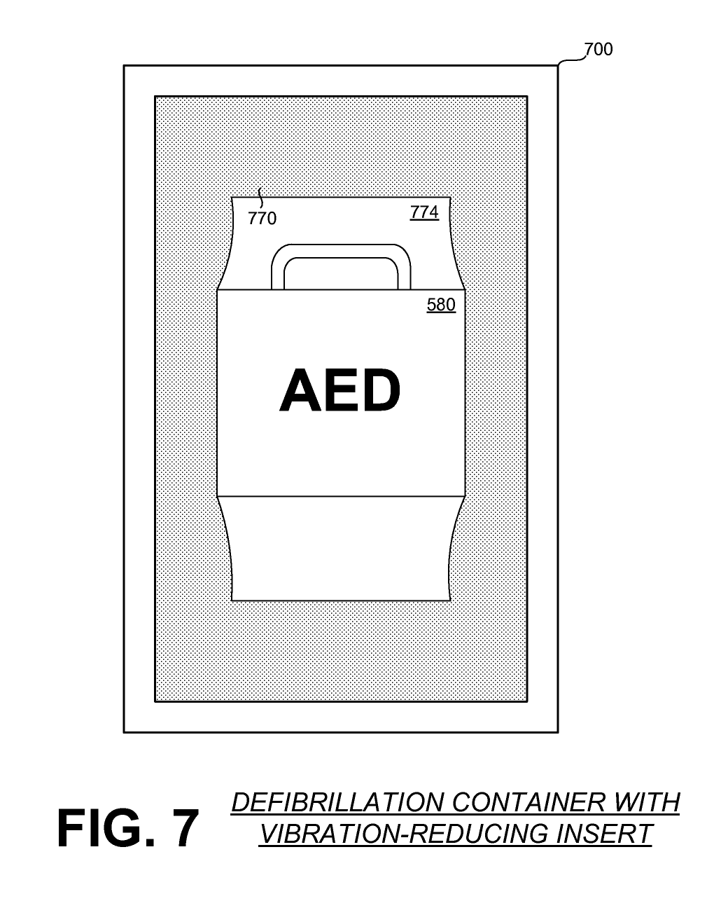

FIG. 7 is another front-sectional view of a defibrillator container shell 700 according to embodiments. Shell 700 includes a vibration dampening material 770 that receives an external defibrillator substantially matingly according to other embodiments. Comparing FIGS. 5 and 7, the embodiment illustrated in FIG. 7 includes a vibration dampening material 770 that substantially fills the container shell 700, except for a material cavity 774. In practice, the defibrillator 580 is placed within the material cavity 774, and is held there by the restorative and friction forces of the vibration dampening material 770 pressing against the housing of the defibrillator 580. Of course, in FIG. 7 the defibrillator 580 could also rest on the bottom portion of the vibration dampening material 770. The material cavity 774 may be shaped to exactly fit the size of the defibrillator 580 stored within, or, as illustrated in FIG. 7, the cavity may be larger than the defibrillator in one or more dimensions.

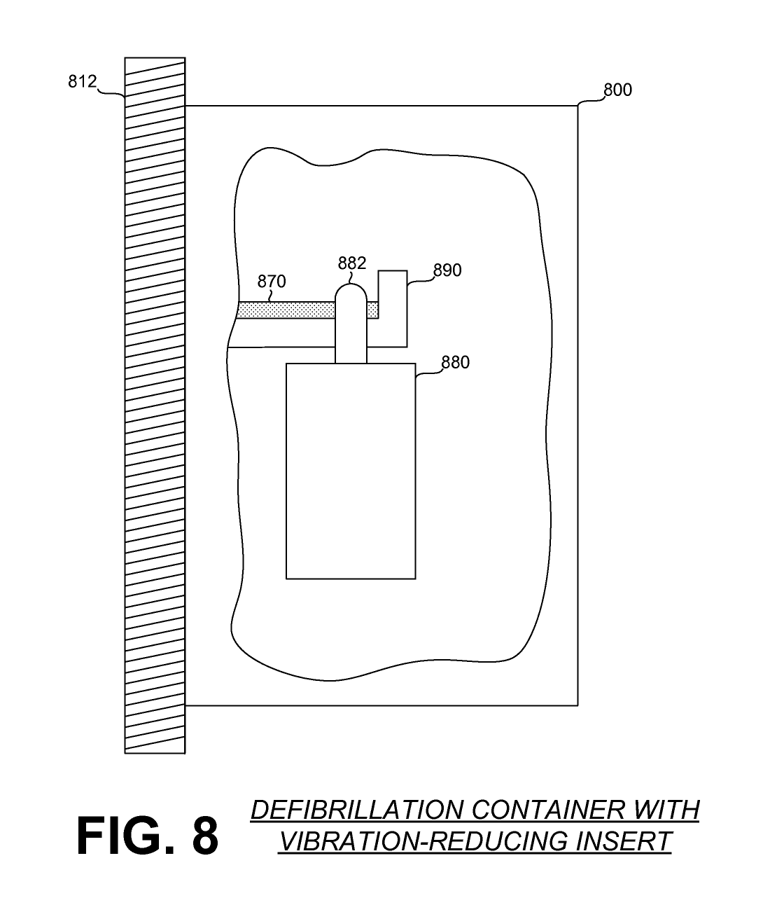

FIG. 8 is a side-sectional view of a defibrillator container 800 mounted on a host structure 812 according to embodiments of the invention. Differently in this embodiment, the container 800 includes a hook 890, upon which a defibrillator 880 may be hung from a handle 882. The hook 890 may be partially or wholly covered by a vibration dampening material 870 so that a lower surface of the handle 882 rests at least partially on the vibration dampening material, and less or not at all on a rigid portion of the hook 890. In such a manner, at least some of the vibration-dampening material 870 is positioned between the hook and the housing of the defibrillator, and is structured to reduce or substantially eliminate vibrations of the container 800 being imparted to the defibrillator 880.

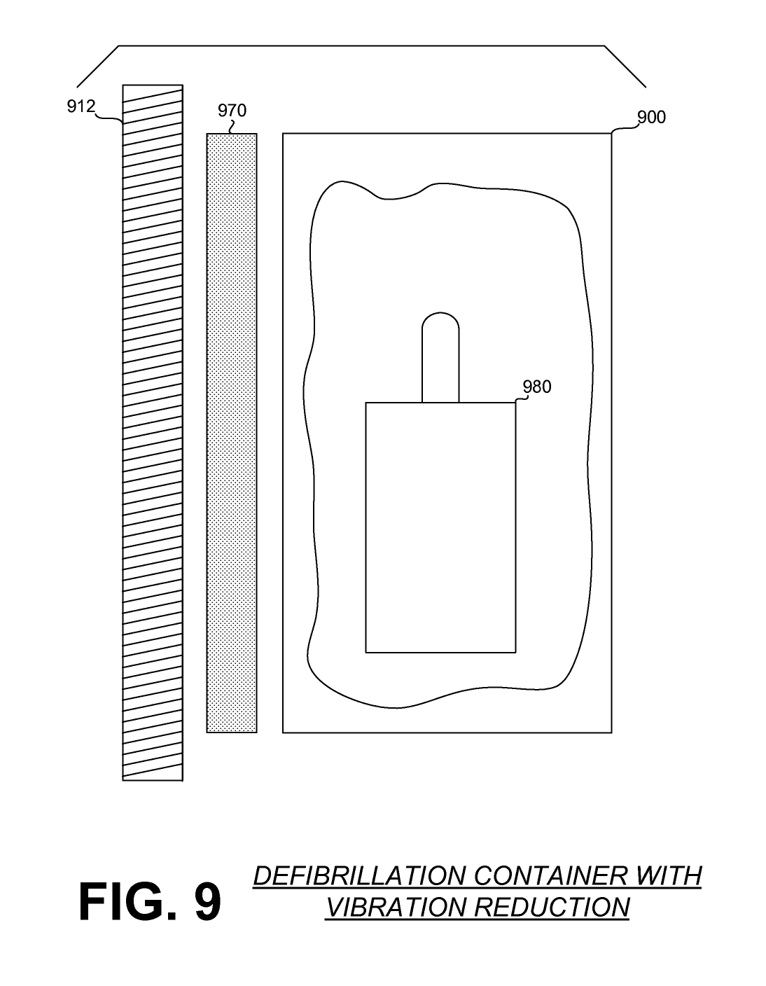

FIG. 9 is a side sectional view of another embodiment of a container shell 900 that stores an external defibrillator 980. In this embodiment, a vibration dampening material 970 is disposed between the container shell 900 and a host structure 912 according to embodiments of the invention. The vibration dampening material 970 may include holes through which fasteners may fasten the container shell to the host structure 912. In this embodiment the container shell 900 and the vibration dampening material 970 are arranged such that vibration from the host structure 912 travels first from the host structure to the vibration dampening material, and then a reduced amount of vibration travels from the vibration dampening material to the container shell. The defibrillator 980 may rest on a surface of the container shell 900 itself or on a hook (not pictured in FIG. 9). In either case the vibration dampening material 970 reduces the amount of vibration imparted to the container shell 900 itself. In this embodiment vibration reduction may be achieved for any device stored in the container shell 900, in addition to the defibrillator 980, such as alarms, communication modules, supplies, etc. This embodiment may be particularly well suited for retro-fitting existing container shells 900 that may not have enough storage room for a vibration dampening material to be stored within the container shell 900.

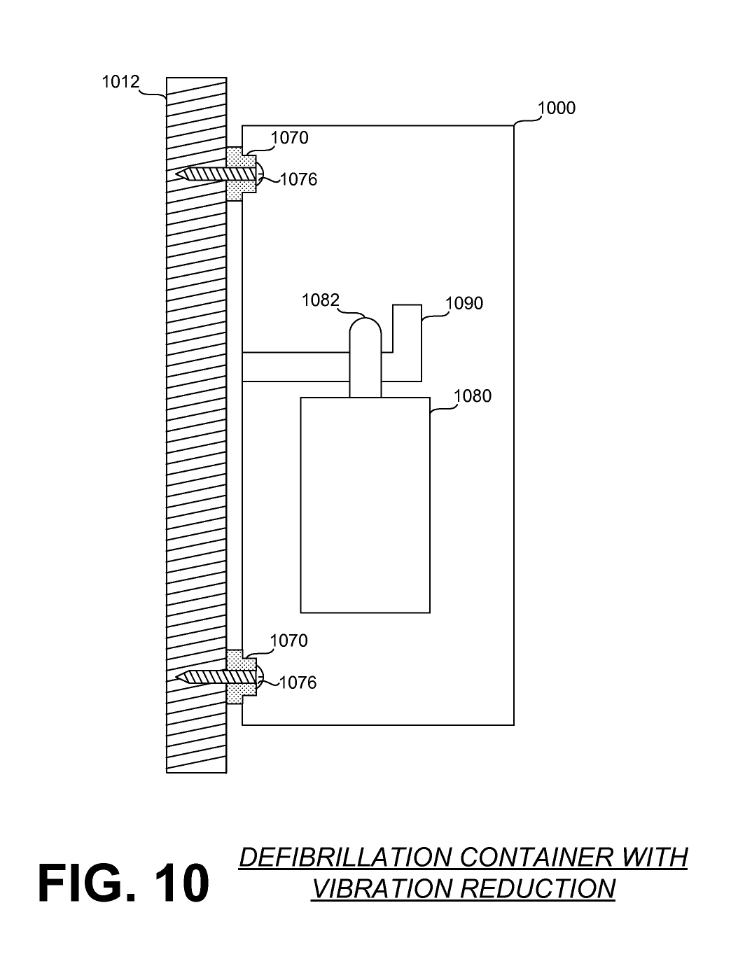

FIG. 10 is a side sectional view of a defibrillator container shell 1000 made according to embodiments of the invention. The container shell 1000 is affixed to a host structure 1012 with a fastener 1076 and grommet formed of a vibration dampening material 1070. In this embodiment the container shell 1000 is held to the host structure 1012 by the fastener 1076 and vibration dampening material in combination. Vibration from the host structure 1012 is imparted to the fastener 1076, but may be diminished by virtue of the vibration dampening material before reaching the container shell 1000. Although FIG. 10 illustrates a defibrillator 1080 being held on a hook 1090 by a handle, 1082, the entire container shell 1000 is isolated or partially isolated from vibration of the host structure 1012. Therefore, the defibrillator 1080 would experience the same vibration reduction if it were stored on a lower surface of the container shell 1000, and not hung from the hook 1090.

In practice, the diagrams illustrated above may be illustrated functionally, and embodiments of the invention operate no matter if the appearance deviates from the views as illustrated. Further, embodiments of the invention may include multiple portions of vibration reducing material in various areas. For example, the hook 1090 of FIG. 10 could also include vibration dampening material disposed thereon. In this way there are at least two areas where vibration dampening could occur, first at the grommet of vibration dampening material 1070, and then again at the vibration dampening material on the hook 1090. Other embodiments may combine, for instance, a vibration material between the container shell and the host structure, such as illustrated in FIG. 9, and additionally include vibration-reducing material within the cabinet, such as the vibration-reducing material 770 illustrated in FIG. 7. The vibration-reducing materials in such a combined system could be made from the same or different materials, or from the same material having different density or restorative properties, for example. This combined system may provide particularly good protection for a broad spectrum of vibration sources. For example, this system may be effective at reducing both high-frequency, low amplitude vibrations such as airplane engine vibration, as well as reducing low-frequency, higher amplitude vibration, such as from airplane turbulence. Other embodiments may include various combinations and sub-combinations of the different vibration-reducing materials and locations described above.

In some embodiments the vibration-dampening material may substantially or partially fill the housing, and the container shell could additionally include a grommet disposed through a mounting hole of the container shell and be structured to receive a fastener therethrough. In other embodiments the container shell could include a hook configured to hold the housing of the defibrillator, a vibration-dampening material positioned between the hook and the housing of the defibrillator, and additionally includes vibration-dampening material between the host structure and the container shell. The vibration dampening material could take the form of a grommet, such as illustrated in FIG. 10, or could take the form of vibration dampening material as illustrated in FIG. 9. Other combinations are also possible.

Other embodiments include combinations or sub-combinations of features described herein, including for example, embodiments that are equivalent to extracting an individual feature from one embodiment and inserting into another embodiment.

In this description, numerous details have been set forth in order to provide a thorough understanding. In other instances, well-known features have not been described in detail in order to not obscure unnecessarily the description.

A person skilled in the art will be able to practice the present invention in view of this description, which is to be taken as a whole. The specific embodiments as disclosed and illustrated herein are not to be considered in a limiting sense. Indeed, it should be readily apparent to those skilled in the art that what is described herein may be modified in numerous ways. Such ways can include equivalents to what is described herein. In addition, the invention may be practiced in combination with other systems.

The following claims define certain combinations and subcombinations of elements, features, steps, and/or functions, which are regarded as novel and non-obvious. Additional claims for other combinations and subcombinations may be presented in this or a related document.

In the claims appended herein, the applicant invokes 35 U.S.C. §112, paragraph 6 only when the words “means for” or “steps for” are used in the claim. If such words are not used in a claim, then the inventor does not intend for the claim to be construed to cover the corresponding structure, material, or acts described herein (and equivalents thereof) in accordance with 35 U.S.C. §112, paragraph 6.

What is claimed is:

1. An apparatus for storing an external defibrillator that has a housing on a host structure that is subject to vibration, the apparatus comprising:
a container shell mountable to the host structure, the container shell structured to store the defibrillator within; and
a vibration-dampening material disposed between the host structure and the housing of the defibrillator, the vibration-dampening material configured to reduce an amount of vibration of the host structure imparted to the defibrillator.

2. The apparatus of claim 1, in which
the container shell and the vibration dampening material are arranged such that the vibration travels from the host structure first to the container shell, and from the container shell to the vibration dampening material.

3. The apparatus of claim 1, in which
the container is affixed to the host structure.

4. The apparatus of claim 1, in which
the vibration-dampening material is positioned between the housing of the defibrillator and the container shell.

5. The apparatus of claim 1, in which
the vibration-dampening material is within the container shell, and
the housing of the defibrillator rests on the vibration-dampening material.

6. The apparatus of claim 1, in which
the vibration-dampening material comprises foam.

7. The apparatus of claim 1, in which
the vibration-dampening material is removable from the container shell.

8. The apparatus of claim 1, in which
the vibration-dampening material includes access passages to mounting holes through which the container shell may be affixed to the host structure.

9. The apparatus of claim 1, in which
the container shell includes a hook configured to hold the housing of the defibrillator and
the vibration-dampening material is positioned between the hook and the housing of the defibrillator.

10. The apparatus of claim 1, in which
the vibration-dampening material partially fills the container shell.

11. The apparatus of claim 1, in which
the vibration-dampening material substantially fills the container shell.

12. The apparatus of claim 11, in which
the vibration-dampening material is in physical contact with the housing of the defibrillator.

13. The apparatus of claim 12, in which
the vibration-dampening material includes a cavity structured to receive the housing of the defibrillator substantially matingly.

14. The apparatus of claim 1, in which
the container shell and the vibration dampening material are arranged such that the vibration travels from the host structure first to the vibration dampening material, and from the vibration dampening material to the container shell.

15. The apparatus of claim 1, in which
the vibration-dampening material comprises a grommet disposed through a mounting hole of the container shell, and
the grommet is structured to receive a fastener therethrough, the fastener structured to secure the container shell to the host structure.

16. An apparatus for storing an external defibrillator that has a housing on a host structure that is configured to travel and is subject to vibration from a propulsion system of the structure when traveling, the apparatus comprising:
a container shell mountable to the host structure while the structure is traveling, the container shell structured to store the defibrillator within; and
a vibration-dampening material disposed between the host structure and the housing of the defibrillator, the vibration-dampening material configured to reduce an amount of vibration of the host structure imparted to the defibrillator.

17. The apparatus of claim 16, in which
the container shell and the vibration dampening material are arranged such that the vibration travels from the host structure first to the container shell, and from the container shell to the vibration dampening material.

18. The apparatus of claim 16, in which
the container is affixed to the host structure.

19. The apparatus of claim 16, in which
the vibration-dampening material is within the container shell, and
the housing of the defibrillator rests on the vibration-dampening material.

20. The apparatus of claim 16, in which
the vibration-dampening material is removable from the container shell.
